(19) United States
(12) Patent Application Publication
(10) Pub. No.: US 2022/0054033 A1
(43) Pub. Date: Feb. 24, 2022

(54) EEG SIGNAL REPRESENTATIONS USING AUTO-ENCODERS

(71) Applicant: X Development LLC, Mountain View, CA (US)

(72) Inventors: Garrett Raymond Honke, Mountain View, CA (US); Pramod Gupta, Mountain View, CA (US); Mustafa Ispir, Mountain View, CA (US); Nina Thigpen, Sunnyvale, CA (US)

(21) Appl. No.: 16/999,636

(22) Filed: Aug. 21, 2020

(51) Int. Cl. A61B 5/048; A61B 5/0478; A61B 5/00

(52) U.S. Cl. CPC A61B 5/048; A61B 5/0478; A61B 5/6814; A61B 5/7203; A61B 5/7264; A61B 5/743

(57) ABSTRACT

Methods, systems, and apparatus, including computer programs encoded on a computer storage medium, for obtaining, from one or more electrodes, electroencephalographic (EEG) signals from a user; generating signal vectors from the EEG signals, each signal vector representing one channel of EEG signals. The actions include providing the signal vectors as input data to a variational autoencoder (VAE), wherein the VAE generates a latent representation of the input data, the latent representation having lower dimensionality than the signal vectors, and reconstructs the latent representation into an event related potential (ERP) of the corresponding EEG signal. The actions include providing, for display to a user, a graphical representation of the ERPs.

### TECHNICAL FIELD

This disclosure generally relates to using neural networks to determine event related potentials from electroencephalography data.

### BACKGROUND

Electroencephalography (EEG) is a method used to detect electrical activity in a user's brain. By affixing electrodes to the scalp of a user and measuring received voltage fluctuations over a period of time, diagnoses can be made related to the neuronal activity detected. Diagnostic applications tend to focus on potential fluctuations that are responsive to an external stimulus or ‘event’. By recording the electrode voltage fluctuations over a period of time and triggering a stimulus event that causes a time-locked fluctuation, an ‘event related potential’ (ERP) can be determined. However, determining an ERP with a high degree of confidence requires careful elimination of stray noise in the signal. Sources of noise can be electrical, environmental, or accidental, including sources internal to the user such as ocular muscle movement, or electrocardiographic noise. This is generally done through time series averaging and can take hundreds, potentially thousands, of recordings to accurately determine an ERP.

### SUMMARY

In general, the disclosure relates to a machine learning system that distinguishes ERP from an input sample of a single patient's EEG signals. In some examples, the system can use a database of training data to create a latent vector representation of varying signal elements. Once the machine learning system has been fed sufficient training data and the latent vector representation determined, the machine learning system can be given input data and reduce the dimensionality to eigenvalues of the latent variable space. During the reduction, the system de-noises the input sample of patient EEG signal to remove signal artifacts not determined to be related to an ERP. Examples of de-noising include removal of endogenous electromagnetic interference from the user (e.g., electro-oculographic signal) unrelated to the ERP. From the trained latent variables, the β-variational encoder machine learning algorithm produces a de-noised EEG reading with reconstructed ERPs of the input EEG signals.

In some implementations, the system disentangles possible sources of variance in the signal such that they can be visualized and inspected for the purpose of identifying and attenuating unique sources of noise—line noise, amplifier disturbance, and blink artifacts and other features of the electroencephalogram. In other words, each unit in the lower-dimensional representation learns a particular latent variable of the EEG (e.g., a pattern of brain activity or stereotyped ERP phenomena). Visualization of this output can help the technician or analyst identify patterns of interest in the EEG signal such as amplitude deflections in critical time windows without the nuisance of background interference and neural processing unrelated to the stimulus of interest.

In some implementations, the visualization is presented as a chart of ERP segments. Each column in the chart can represent a particular latent variable of the EEG (e.g., a type of brain activity or EEG phenomena). Each row of the chart can represent an incremental scaling or perturbation of the latent variables. The chart can aid a clinician understand which latent variables may be contributing to a sample EEG reading. The lower dimensional representation can also be systematically altered to construct novel EEG segments.

In general, innovative aspects of the subject matter described in this specification can be embodied in methods that include the actions of obtaining, from one or more electrodes, electroencephalographic (EEG) signals from a user. The actions include generating signal vectors from the EEG signals, each signal vector representing one channel of EEG signals. The actions include providing the signal vectors as input data to a variational autoencoder (VAE), where the VAE generates a latent representation of the input data, the latent representation having lower dimensionality than the signal vectors, and reconstructs the latent representation into an event related potential (ERP) of the corresponding EEG signal. The actions include providing, for display to a user, a graphical representation of the ERPs. Other implementations of this aspect include corresponding systems, apparatus, and computer programs, configured to perform the actions of the methods, encoded on computer storage devices.

These and other implementations can each optionally include one or more of the following features.

In some implementations, the latent representation includes a set of latent signal vectors, each latent signal vector being generated from a respective one of the signal vectors, and wherein each latent signal vector has lower dimensionality than its respective signal vector.

In some implementations, the latent representation includes a set of latent signal vectors, each latent signal vector being generated from a respective set of two or more signal vectors.

In some implementations, the actions include converting signal vectors from time-domain to frequency-domain prior to providing the signal vectors as input data to the VAE.

In some implementations, the signal vectors are a first set of signal vectors and the action of generating the signal vectors from the EEG signals includes generating, from the first set of signal vectors, a second set of signal vectors that are a duplicate of the first set of signal vectors, and converting the second set of signal vectors from time-domain to frequency-domain, and wherein providing the first set and the second set of signal vectors as input data to the VAE.

In some implementations, the VAE is a βVAE.

In some implementations, a corruption function of the VAE is a salt and pepper, Gaussian, or masking function.

In some implementations, a loss function of the VAE is a forward/reverse KL divergence model.

In some implementations, the VAE performs separable or non-separable convolutions on the input data.

In some implementations, the actions include providing for display, a graphical user interface including at least a first and a second graph, the first graph representing a first latent variable of the EEG signals and the second graph representing a second, different latent variable of the EEG signals; and a control input associated with each of the first and second latent variable, wherein adjustment of the control input causes the VAE to adjust its numerical distribution model for the respective latent variable.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1A is a schematic diagram depicting the gathering of a user's EEG data.

FIG. 1B is a chart depicting an example of collected EEG data.

FIG. 1C is a block diagram depicting an example data file used to hold collected electroencephalography data.

FIG. 2 is a block diagram depicting an example system used to distinguish event related potentials within a user's EEG data.

FIG. 3 is an exemplary graphical user interface showing an output ERP and control inputs to modify the ERP signal based on a numerical model mapping.

FIG. 4 is a flow chart of an example process for distinguishing event related potentials within a user's EEG data.

FIG. 5 depicts a schematic diagram of a computer system that may be applied to any of the computer-implemented methods and other techniques described herein.

Like reference numbers and designations in the various drawings indicate like elements.

### DETAILED DESCRIPTION

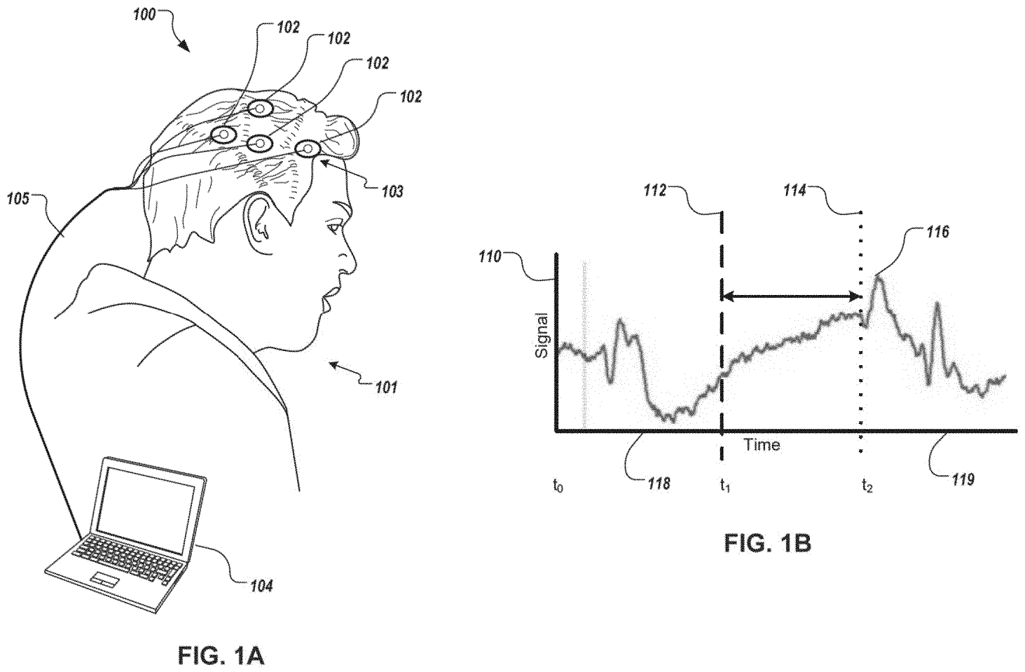

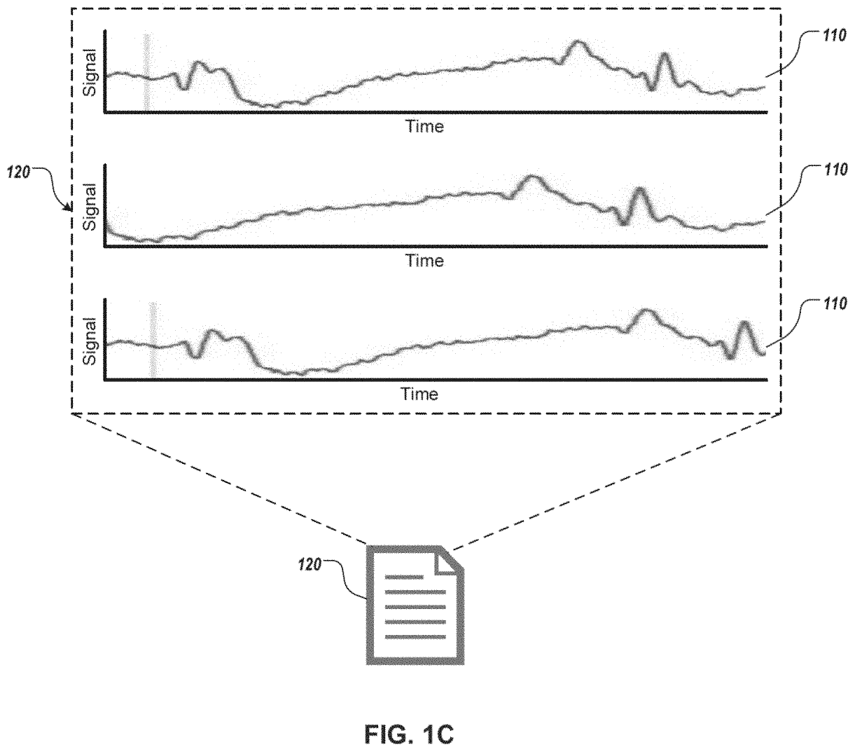

FIG. 1A is a schematic diagram of an electroencephalographic (EEG) system 100 for the collection of EEG data from a user 101. One or more electrodes 102 can be affixed to the scalp 103 of a user 101. In some implementations, the one or more electrodes can include two or more electrodes, e.g., two or more, 10 or more, 20 or more, 40 or more, 80 or more, 100 or more electrodes. In some implementations, the electrodes 102 can be held in place by an adhesive or can be affixed to an article designed to be worn by the user which can hold the electrodes against the scalp 102 of a user 101. In some implementations, the electrodes can be affixed invasively within the scalp 102 of the user 101.

The electrodes 102 can be connected to a receiving computer system 104. For example, the electrodes 102 can be connected to the receiving computer system 104 by a wired connection 105 or by a wireless connection.

While connected to the receiving computer system 104, the electrodes 102 can detect neurological electrical signals from the scalp 102 of the user 101 and transmit the electrical signals to the receiving computer system 104, e.g., as electrode signals 110. Each electrode signal 110 can correspond to an electrode 102 affixed to the scalp 103 of a user 101, and represents an individual EEG channel. In general, there can be as many electrode signals 110 as there are electrodes 102.

An example of a transmitted electrode signal 110 from a single electrode is shown in FIG. 1B. The y-axis can show the value of the electric signal while the x-axis can show the time over which the electric signal was collected. There can be more than one signal component to a collected electrode signal 110. Signal components of an electrode signal 110 can include, but are not limited to, systematic, accidental, and stimulated components. For example, systemic components can include 60 Hz line noise, and accidental components can include unintended motion of the user causing the recording equipment to produce signal artifacts. There can also be endogenous components—components that are biologically generated unnecessary for the purpose of measuring neuronal electrical activity. Examples of such endogenous components include electrical activity from muscle movements (i.e., the electro-oculogram) and cardiographic activity.

An ERP 116 can include a stimulated component, e.g., a signal that is related to an external user stimulus. In some implementations, the external user stimulus can be administered by a clinician in a clinical setting. For example, the external user stimulus can be a sensory stimulation such as a flash of light, or a loud noise. In further examples, the external user stimulus can be a motor event such as a button press, or an eye movement. In further examples, the external user stimulus can be a mental operation such as an anticipation, or recall of mental imagery.

In some implementations, the EEG signals are obtained during an EEG “trial” during which a user is presented with various stimuli to evoke particular ERPs 116. For example, a stimulus intended to trigger particular responses in portions of the brain, such as the visual cortical system, or the anterior cingulate cortex, can be presented to a user and the corresponding electrical activity recorded in the EEG trial can be marked or labeled, and associated with a timestamp of when the stimulus was presented.

The stimulus can include, but is not limited to, visual content such as images or video, audio content, interactive content such as a game, or a combination thereof. For example, emotional content (e.g., a crying baby; a happy family) can be configured to probe the brain's response to emotional images. As another example, visual attentive content can be configured to measure the brain's response to the presentation of visual stimuli. Visual attentive content can include, e.g., the presentation of a series of images that change between generally positive or neutral images and negative or alarming images. For example, a set of positive/neutral images (e.g., images of a stapler, glass, paper, pen, glasses, etc.) can be presented with a negative/alarming image (e.g., a frightening image) interspersed there between. The images can be presented randomly or in a pre-selected sequence. Moreover, the images can alternate or “flicker” at a predefined rate.

As another example, error monitoring content can be used to measure the brain's response to making mistakes. Error monitoring content can include, but is not limited to, interactive content designed to elicit decisions from a patient in a manner that is likely to result in erroneous decisions. For example, the interactive content can include a test using images of arrows and require the patient to select which direction the arrow(s) is/are pointing, but may require the decisions to be made quickly so that the user will make errors. In some implementations, no stimuli are presented, e.g., in order to measure the brain's resting state to obtain resting state electrical activity.

While recording the electrode signal 110, an external user stimulus can be administered at a time t₁ 112. The electrode signal 110 can be collected long enough to see a response at a separate time t₂ 114. The recorded electrode signal 110 can have a pre-stimulus signal 118 and a post-stimulus signal 119. The pre-stimulus signal 118, e.g., a baseline, can be the signal recorded in the region between the beginning of the recorded electrode signal 110 at to and the time at which the stimulus is induced at t₁ 112. The post-stimulus signal 119 can be the signal recorded after the induced response is recorded at t₂ 112. The ERP 116 can consist of a series of positive and negative voltage deflections from the pre-stimulus signal 118. The ERP 116 can also be compared to the post-stimulus signal 119 to determine voltage deflections from the post-stimulus signal 119.

The pre-stimulus signal 118 and the post-stimulus signal 119 can also be used to compare systematic and accidental components in the EEG system 100, e.g., unintended motion.

As shown in FIG. 1C, once the electrode signals 110 have been collected from the electrodes 102 for a time period, the receiving computer system 104 can then combine one or more electrode signals 110 into an EEG data file 120. FIG. 1C depicts 3 exemplary electrode signals 110 being combined into an EEG data file 120 but in general, one or more electrode signals 110 can be combined into an EEG data file 120. In some embodiments, there can be as many electrode signals 110 as there are electrodes 102 combined into the EEG data file 120.

Once the electrode signals 110 have been collected by the receiving computer system 104 and joined into an EEG data file 120, the EEG data file 120 can be sent to an EEG processing system 200 for processing using a machine learning model.

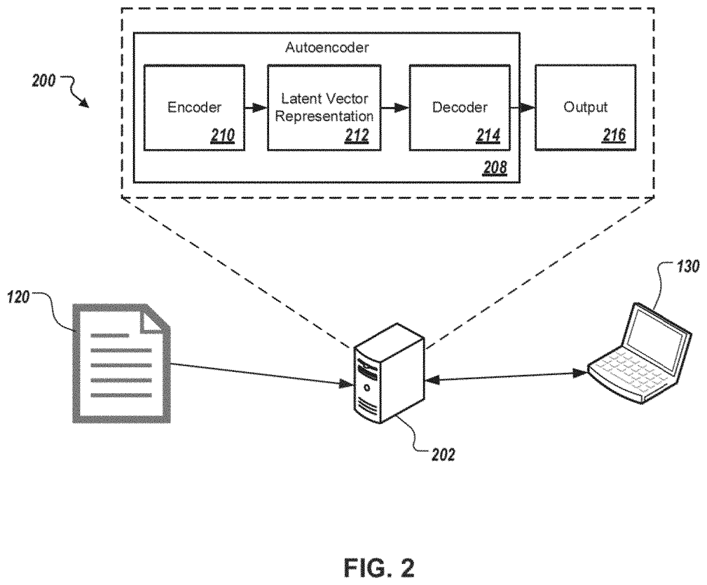

Referring to FIG. 2, the EEG processing system 200 can include a system of one or more computers 202. The EEG processing system 200 can be configured to determine a latent vector representation of EEG data files 120 and output de-noised ERPs 116. For example, the EEG processing system 200 can store and execute one or more machine learning engines that are programed to determine a latent vector representation of collected EEG data and output de-noised ERPs. More specifically, the EEG processing system 200 can include one or more machine learning models that have been trained to receive model inputs (e.g., user EEG data) and generate an output based on the received model input.

In some implementations, the machine learning model can be a neural network. A neural network is a machine learning model that includes one or more input layers, one or more output layers, and one or more hidden layers that each apply a transformation to a received input to generate an output. In some implementations, the neural network may be an autoencoder 208. In some implementations, the neural network may be a variational autoencoder (VAE). In some implementations, the neural network may be a β-variational autoencoder (βVAE).

As shown in FIG. 2 and described above, the EEG processing system 200 can include an autoencoder 208. An autoencoder 208 is a neural network designed to construct a latent vector representation 212 of an input in an unsupervised way. By using an encoding engine 210 to compress the electrode signals 110 within a received EEG data file 120 into a latent vector representation 212 it can greatly reduce the dimensionality of the input electrode signals 110. The latent vector representation 212 can include one or more hidden variables with which the autoencoder 208 has been trained to represent components in input electrode signals 110. The hidden variables within the latent vector representation 212 can have one or more scores representing components of the electrode signals 110 on which the autoencoder 208 has been trained. In general, the dimensionality of the input electrode signals 110 can be larger than the dimensionality of the latent vector representation 212.

The encoding engine 210 can have one or more input layers to receive the electrode signals 110 within a received EEG data file 120. In general, the encoding engine 210 can process the electrode signals 110 contained within the EEG data file 120 individually or in parallel. In general, the encoding engine 210 can process the data contained in electrode signals 110 or it can apply one or more modifying functions. In some implementations, the encoding engine 210 can duplicate one or more input electrode signals 210 before applying a modifying function, e.g., a domain transformation function, or a corruption function.

In some implementations, the encoding engine can apply a corruption function to partially sample the electrode signals 110. The corruption function can include using a sampling distribution to partially sample the electrode signals 110. The sampling distribution can include a Gaussian distribution, masking distribution, salt-and-pepper distribution, or other distributions.

In some implementations, the encoding engine 210 can apply a domain transformation function. For example, the encoding engine 210 can transform data points collected across time, e.g., time domain, into data points correlated across frequency, e.g., frequency domain. In some implementations, the encoding engine 210 can perform this transformation with a Fourier transform function. In some implementations, the encoding engine 210 can perform this transformation with a fast Fourier transform (FFT) function.

In some implementations, the encoding engine 210 can perform a domain transformation before using a corruption function to partially sample the input electrode signals 210. In some implementations, the encoding engine 210 can use a corruption function and then perform a domain transformation.

In operation, the encoding engine 210 can process the electrode signals 210 by performing convolutions across the time points of the input electrode signals 210. Convolutions of the electrode signals 210 can be separable or they can be non-separable. For example, separable convolutions can be performed within single electrode signals 210 and non-separable convolutions can be performed between two or more input electrode signals 210 that take cross-electrode activity into account in the activation function.

After the encoding engine 210 has determined the latent vector representation 212 of the received electrode signals 110, the autoencoder 208 can use a decoding engine 214 to construct an output 216 from the latent vector representation 212. In general, the decoding engine can be one or more hidden layers within the autoencoder 208 neural network. The autoencoder 208 can execute the decoding engine 214 to attempt to construct an output 216 that is similar to the input electrode signals 110. Once the autoencoder 208 has constructed an output 216, the autoencoder 208 can then use a loss function to determine the difference, e.g., loss, between the output 216 and the input electrode signals 110. For example, the loss function an autoencoder 208 uses can be a mean squared error (MSE) function or MSE plus a K-L divergence loss function. The K-L divergence loss function can be a forward K-L divergence loss function or a reverse K-L divergence loss function.

After determining the loss between an output 216 and the input electrode signal 110, the autoencoder 208 can update one or more variables within the latent vector representation 212 to attempt to reconstruct the output 216 to achieve a lower loss. If a lower loss is achieved, the autoencoder will continue to process electrode signals 110 with the updated latent vector representation 212. The updated latent vector representation 212 can then be used to encode and decode one or more electrode signals 110 before calculating a new loss. In this manner, the autoencoder 208 can be trained to reconstruct input electrode signals 110 from the latent vector representation with the lowest loss.

As the autoencoder 208 is trained on input electrode signals 110 that include one or more components, the autoencoder 208 will be trained to represent one or more components within the latent vector representation 216. For example, if the input electrode signals 110 have ERPs 116, the autoencoder 208 can be trained to represent ERPs 116 in the latent vector representation 216.

In some implementations, the autoencoder 208 can be trained to represent one input electrode signal 110 within the latent vector representation 216. In some implementations, the autoencoder 208 can be trained to represent a set of input electrode signals 110 within a latent vector representation 216, e.g., more than one electrode signal 110.

An autoencoder 208 trained on electrode signals 110 with ERPs 116 can output reconstructed electrode signals 110 displaying only the reconstructed ERPs 116 using the variable associated with ERPs 116 from the latent vector representation 212. In some implementations, the autoencoder 208 trained on electrode signals 110 with ERPs 116 can further output the latent vector representation 212 to allow filtering along one or more latent vector representation 212 variable scores.

The output 216 generated by the autoencoder 208 can then be sent to a clinician device 130 or to a user 101 device. Examples of the clinician device 130 or user device can include desktop computers, laptop computers, smart phones, or tablet computers.

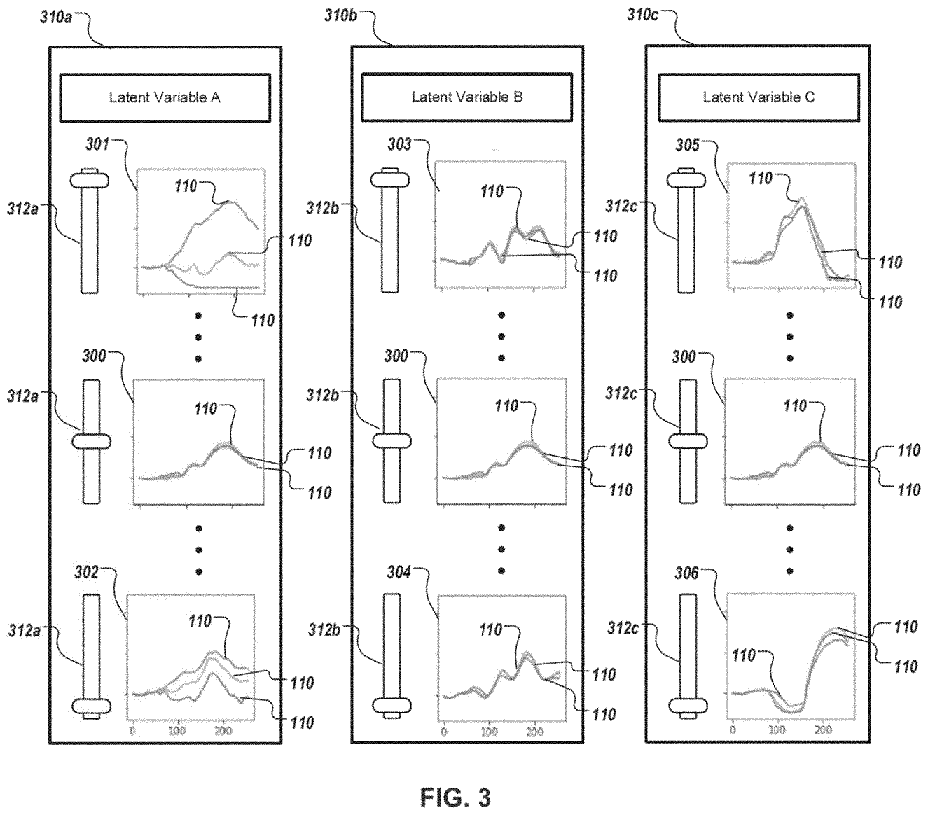

In general, the output 216 can be an interactive representation of the reconstructed ERP. The representation can be filtered based on the latent vector representation 212 variable scores for the ERP. The filter can be manually controlled by the clinician or user to display the filtered ERP along one or more latent vector representation 212 variable scores. An example of the interactive representation output 216 can be seen in FIG. 3. The output 216 can be an ERP interactive graph 300 depicting the fully denoised ERP in every electrode signal 110. Along with the ERP graph 300, the autoencoder 208 can output a numerical model which can be used to map the latent vector representation 212 of the EEG data files 120 to the output ERP graph 300.

The ERP graph 300 can be displayed on the clinician device 130 in a graphical user interface (GUI) 310 format. The GUI 310 can include one or more control inputs 312 to receive input from the clinician on the clinician device 130. In some implementations, the GUI 310 can performed automated functions before displaying the ERP graph 300 to the clinician device 130, such as filtering or scaling one or more GUI interfaces. The GUI can be respondent to touch or to computer input devices, e.g., mouse, trackball, or keyboard.

For each variable within the latent vector representation 212, there can be a corresponding control input 312. FIG. 3 depicts three example control inputs 312 for Latent Variables A, B, and C as depicted by GUI interfaces 310a, 310b, and 310c, respectively. The control inputs 312a, 312b, 312c for the GUI interfaces 310a, 310b, and 310c are depicted as sliders but they can be any control input 312 used in GUI interfaces such as radio buttons, numerical input boxes, or tuner dials.

The control input 312 for each latent variable can scale the electrode signals 210 that make up the ERP graph 300 by filtering the latent vector representation 212 variable scores for each electrode channel 110 within the ERP graph 300. The control inputs 312 can map a value between a normalized maximum and a normalized minimum value that allows a clinician or user to choose the level of latent vector representation 212 variable scores filtering applied to the ERP graph 300. For example, latent variable A control input 312a is shown in three positions, corresponding to a normalized maximum filtration value, a zero filtration value, and a normalized minimum filtration value, respectively. These three positions and their corresponding filtration values can result in graphs 301, 300, and 302, from top to bottom.

The top ERP graph 301 depicts control input 312a set to a normalized maximum value which can apply the maximum mapping of the numerical distribution model for latent variable A. It can be seen in ERP graph 301 that the latent vector representation 212 variable scores filtering can affect each displayed electrode signal 110 individually. In contrast, the ERP graph 302 depicts control input 312a set to a normalized minimum value which can apply the minimum mapping of the numerical distribution model for latent variable A. In general, this can result in a different displayed graph from the maximum mapping shown in 301 and 300 where no mapping is applied.

FIG. 3 additionally shows GUI interfaces 310b, and 310c for two additional latent variables B and C. The control inputs 312b and 312c can control the numerical distribution model mapping for latent variables B and C. Because latent variables B and C can be separable from latent variable A, when control inputs 312b and 312c are set to respective maximum and minimum mapping values, as shown in graphs 303, 304, 305 and 306, the graphs are independent of the changes shown in graphs 301 and 302.

In some implementations, the GUI 310 can allow the clinician to select and display ERP graphs from one or more users. This can allow comparisons of one or more latent variables between the one or more users, or groups of users.

In some implementations, the control inputs 312 can be used to further train the autoencoder 208. For example, a clinician can use the control input 312a to change the numerical distribution model mapping for latent variable A to a different normalized value. The clinician can then use the clinician device 130 to transmit the new numerical distribution model mapping for latent variable A to further train the autoencoder 208 to represent latent variable A within the latent variable representation 212.

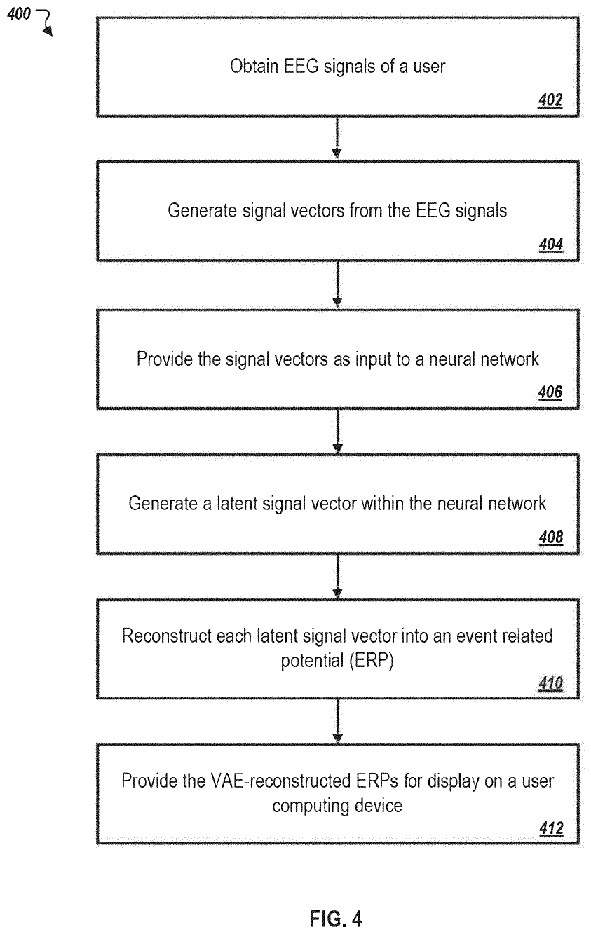

FIG. 4 depicts a flowchart of an example process 400 for de-noising EEG data in accordance with implementations of the present disclosure. In some implementations, the process 400 can be provided as one or more computer-executable programs executed using one or more computing devices. In some examples, process 400 is executed by one or more machine learning models.

The system obtains user electroencephalographic data from one or more electrodes affixed to the scalp of the user (402). Each electrode can provide to the system analog measurements of the neurological electrical signals of a user as an electrode channel. The signal of each electrode channel can be a series of electric potential amplitude measurements collected at a regular time frequency. The system can convert these time-series analog measurements to a digital file by sampling the analog signal across a set time period, converting the electric potential amplitude measurements to a digital signal, and concatenating the digital signals into a digital file. This process can be repeated for the number of electrode channels provided such that the final digital file contains every electrode channel. The digital file can then be transmitted to the EEG processing system.

Upon transmission of the digital file, the EEG processing system can use an encoder neural network to generate signal vectors from the EEG signals (404). The signal vectors can represent one, more than one, or a portion of an EEG signal. The EEG signals can be duplicated before generation of the signal vectors. The EEG processing system can then optionally apply a modifying function, such as a corruption function or a domain transformation function, to the transmitted or duplicated EEG signal vectors.

The signal vectors can then be provided as input to a neural network (406), e.g., a variational, or a β-variational autoencoder. The dimensionality of the input signal vectors can then be reduced by the neural network through separable or non-separable convolutions across the domain of the data, e.g., time, or frequency. The autoencoder can be trained to reduce the dimensionality of the signal vectors to generate a latent vector representation where each variable in the vector represents one or more components of the input signal vectors (408). When the encoder has reduced the dimensionality of the signal vectors into a latent vector representation, the EEG processing system can use a decoding engine to construct an output. The output can be an attempted representation of the input signal vectors.

The EEG processing system can then use a loss function to compare the output to the input signal vector and determine a difference, e.g., loss. The EEG processing system can then use the loss to update the latent vector representation. With this updated latent vector representation, the autoencoder can construct an updated output and determine a new loss. If the new loss is a lower value than the original loss, the autoencoder will store the updated latent vector representation to reconstruct further encoded signal vectors.

The latent vector representation can include variables for one or more components of the input signal, e.g., systematic, accidental, or stimulated. For example, stimulated components, e.g., ERPs, can be temporally related to external user stimulus, e.g., a sensory stimulation, motor event, or mental operation. The decoding engine can reconstruct one or more components of the latent vector representation into an output signal vector. For example, the decoding engine can reconstruct the latent vector representation variable using only the variable related to an ERP (410). During autoencoder reconstruction of the latent vector representation, non-stimulated components can be attenuated leaving only the ERP in the reconstructed output.

The system can then transmit the reconstructed ERP over a network to a user device thereby providing the neural network-reconstructed ERPs for display on a user computing device (412). The user can then interact with a graphical user interface which displays the ERP alongside one or more control inputs, each relating to a variable stored within the latent vector representation. The user may then use at least one control input to re-scale the latent variable represented in the ERP.

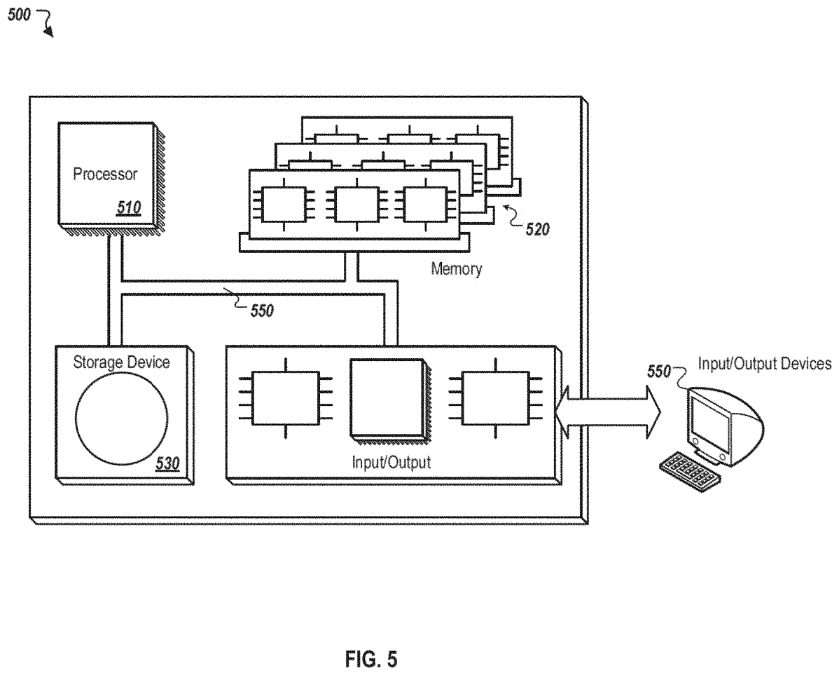

FIG. 5 is a schematic diagram of a computer system 500. The system 500 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 500) and their structural equivalents, or in combinations of one or more of them. The system 500 is intended to include various forms of digital computers, such as laptops, desktops, workstations, personal digital assistants, servers, blade servers, mainframes, and other appropriate computers, including vehicles installed on base units or pod units of modular vehicles. The system 500 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

For convenience, implementations of the present disclosure have been discussed in further detail with reference to an example medical context. More specifically, the example context includes predicting the spread of a contagion (e.g., an illness). It is appreciated, however, that implementations of the present disclosure can be realized in other appropriate contexts (e.g., predicting the spread of ideas, social trends, word-of-mouth advertising, etc.).

What is claimed is:

1. A computer-implemented method to de-noise electroencephalographic data, the method comprising:
obtaining, from one or more electrodes, electroencephalographic (EEG) signals from a user;
generating signal vectors from the EEG signals, each signal vector representing one channel of EEG signals;
providing the signal vectors as input data to a variational autoencoder (VAE), wherein the VAE generates a latent representation of the input data, the latent representation having lower dimensionality than the signal vectors, and reconstructs the latent representation into an event related potential (ERP) of the corresponding EEG signal; and
providing, for display to a user, a graphical representation of the ERPs.

2. The method of claim 1, wherein the latent representation comprises a set of latent signal vectors, each latent signal vector being generated from a respective one of the signal vectors, and wherein each latent signal vector has lower dimensionality than its respective signal vector.

3. The method of claim 1, wherein the latent representation comprises a set of latent signal vectors, each latent signal vector being generated from a respective set of two or more signal vectors.

4. The method of claim 1, further comprising converting signal vectors from time-domain to frequency-domain prior to providing the signal vectors as input data to the VAE.

5. The method of claim 1, wherein the signal vectors are a first set of signal vectors,
wherein generating the signal vectors from the EEG signals comprises:
generating, from the first set of signal vectors, a second set of signal vectors that are a duplicate of the first set of signal vectors; and
converting the second set of signal vectors from time-domain to frequency-domain, and
wherein providing the first set and the second set of signal vectors as input data to the VAE.

6. The method of claim 1, wherein the VAE is a βVAE.

7. The method of claim 1, wherein a corruption function of the VAE is a salt and pepper, Gaussian, or masking function.

8. The method of claim 1, wherein a loss function of the VAE is a forward/reverse KL divergence model.

9. The method of claim 1, wherein the VAE performs separable or non-separable convolutions on the input data.

10. The method of claim 1, further comprising providing, for display, a graphical user interface comprising:
at least a first and a second graph, the first graph representing a first latent variable of the EEG signals and the second graph representing a second, different latent variable of the EEG signals; and
a control input associated with each of the first and second latent variable, wherein adjustment of the control input causes the VAE to adjust its numerical distribution model for the respective latent variable.

11. A system comprising:
at least one processor; and a data store coupled to the at least one processor having instructions stored thereon which, when executed by the at least one processor, causes the at least one processor to perform operations comprising:
obtaining, from one or more electrodes, electroencephalographic (EEG) signals from a user;
generating signal vectors from the EEG signals, each signal vector representing one channel of EEG signals;
providing the signal vectors as input data to a variational autoencoder (VAE), wherein the VAE generates a latent representation of the input data, the latent representation having lower dimensionality than the signal vectors, and reconstructs the latent representation into an event related potential (ERP) of the corresponding EEG signal; and
providing, for display to a user, a graphical representation of the ERPs

12. The system of claim 11, wherein the latent representation comprises a set of latent signal vectors, each latent signal vector being generated from a respective one of the signal vectors, and wherein each latent signal vector has lower dimensionality than its respective signal vector.

13. The system of claim 11, wherein the latent representation comprises a set of latent signal vectors, each latent signal vector being generated from a respective set of two or more signal vectors.

14. The system of claim 11, wherein the VAE performs separable, or non-separable convolutions on the input data.

15. The system of claim 11, wherein the operations further comprise providing, for display, a graphical user interface comprising:
at least a first and a second graph, the first graph representing a first latent variable of the EEG signals and the second graph representing a second, different latent variable of the EEG signals; and
a control input associated with each of the first and second latent variables, wherein adjustment of the control input causes the VAE to adjust its numerical distribution model for the respective latent variable.

16. A non-transitory computer readable storage medium storing instructions that, when executed by at least one processor, cause the at least one processor to perform operations comprising:
obtaining, from one or more electrodes, electroencephalographic (EEG) signals from a user;
generating signal vectors from the EEG signals, each signal vector representing one channel of EEG signals;
providing the signal vectors as input data to a variational autoencoder (VAE), wherein the VAE generates a latent representation of the input data, the latent representation having lower dimensionality than the signal vectors, and reconstructs the latent representation into an event related potential (ERP) of the corresponding EEG signal; and
providing, for display to a user, a graphical representation of the ERPs.

17. The medium of claim 16, wherein the latent representation comprises a set of latent signal vectors, each latent signal vector being generated from a respective one of the signal vectors, and wherein each latent signal vector has lower dimensionality than its respective signal vector.

18. The medium of claim 16, wherein the latent representation comprises a set of latent signal vectors, each latent signal vector being generated from a respective set of two or more signal vectors.

19. The medium of claim 16, wherein the VAE performs separable, or non-separable convolutions on the input data.

20. The medium of claim 16, wherein the operations further comprise providing, for display, a graphical user interface comprising:
at least a first and a second graph, the first graph representing a first latent variable of the EEG signals and the second graph representing a second, different latent variable of the EEG signals; and
a control input associated with each of the first and second latent variables, wherein adjustment of the control input causes the VAE to adjust its numerical distribution model for the respective latent variable.
